(19) United States
(12) Patent Application Publication
(10) Pub. No.: US 2011/0224562 A1
(43) Pub. Date: Sep. 15, 2011

(54) THERMOGRAPHIC PLATE FOR THE DETECTION OF TRACES OF BLOOD FLOW IN VESSELS AT DIFFERENT DEPTH

(76) Inventor: Daniele Montruccoli Salmi, Bologna (IT)

(21) Appl. No.: 12/998,585

(22) PCT Filed: Nov. 2, 2009
(86) PCT No.: PCT/EP2009/064470

§ 371 Date: May 5, 2011

(30) Foreign Application Priority Data
Nov. 7, 2008 (IT) BO2008A000673

(51) Int. Cl. A61B 5/026

(52) U.S. Cl. 600/504

(57) ABSTRACT

A method for selective detection, with a thermographic plate, of traces of blood flow that differ from traces with a correct course in vessels at different depths, particularly of the breast, which consists in that a sequence of thermographic images is taken which are acquired by a thermographic plate gently pressed against the skin, which is cooled before each photograph with an air jet whose duration is correlated to the depth of the portion of vessel being investigated, the traces of vessels with a correct course having a substantially centripetal direction with respect to the breast and tapering toward the center of the organ so as to confirm a temperature of the blood flow which decreases because the vessel gradually branches and because the depth from the surface of the skin increases.

The present invention relates to a method for selective detection, with a thermographic plate, of traces of blood flow that differ from traces with correct course in vessels at different depths, particularly of the breast, and to an apparatus for performing the method and for generating thermographic maps of blood flow.

### BACKGROUND OF THE INVENTION

Apparatuses for thermographic investigation of the breast are known which provide the blowing of cold air onto the breast prior to placing thereon the thermographic plate in order to eliminate the influence of the surface heat of the mammary gland: this type of investigation seeks areas where an increase in heat occurs due to the presence of new blood vessels generated within the organ to supply a pretumoral or tumoral lesion: with this type of examination, a series of photographs, preferably of the digital type, of the thermographic plate placed with light pressure against the breast is taken, and the surface of the breast is cooled before each placement operation with a jet of air of a certain width, which strikes almost all of the mammary surface: the obtained images are viewed for identifying the presence, at different depths, of light-colored areolas (calcifications) that are denser, small and substantially circular (not elongated) in shape, which have been demonstrated to be indicators which are always present in precancerous or cancerous lesion situations.

As the investigated case series became longer, it has been found that with small modifications to the apparatus for thermographic investigation of the breast with cooling by blowing air it is possible to investigate the blood flow in breast vessels at different depths and to produce a map thereof without injecting contrast liquids and to identify, considerably in advance with respect to other investigation techniques, the traces of the vessels whose blood flow-rate differs from the norm and is an indicator of tumoral or pre-tumoral formations.

This is in agreement with the literature in the field, which teaches that for breast cancer, since there are no preventive measures or tests that can predict its onset, the only indicator to be sought for early diagnosis is observation of the onset of variations (increase) in blood inflow, such variations being necessary to supply the region where the tumor will appear, even before it is detectable as such.

### SUMMARY OF THE INVENTION

The aim of the present invention is to provide a method and an apparatus which, without the introduction of contrast liquids, allow respectively the selective detection of traces of breast blood circulation in vessels at different depths and performance of the method for generating blood flow maps.

Within this aim, an object of the present invention is to provide a method and an apparatus which are simple, relatively easy to provide in practice, safe to use, effective in operation, and have relatively low costs.

This aim and this and other objects which will become better apparent hereinafter are achieved by the present method for selective detection, with a thermographic plate, of traces of blood flow that differ from traces with correct course in vessels at different depths, particularly of the breast, characterized in that a sequence of thermographic images is taken which are obtained by a thermographic plate gently pressed against the skin, which is cooled before each image with an air jet whose duration is correlated to the depth of the portion of vessel being investigated, the traces of vessels with a correct course having a substantially centripetal direction with respect to the breast and tapering toward the center of the organ so as to confirm a temperature of the blood flow which decreases because the vessel gradually branches and because the depth from the surface of the skin increases.

The apparatus for performing the method for selective detection, with a thermographic plate, of traces of blood flow that differ from traces with correct course comprises a stand provided with a handle which has, at its two ends, a frame for the snug insertion of a thermographic plate and supporting means for a camera, characterized in that it comprises a cooling fan, which is associated with such stand and is mounted and actuated so that it can rotate in a duct whose outlet has a reduced area and is located at the base and at the center of said frame, the selective succession of the images acquired after the respective jets being considered independently of the colors assumed by the plate and as a function of the geometry of the traces suitable to highlight the variation of blood flow-rate in said vessel.

### BRIEF DESCRIPTION OF THE DRAWINGS

Further characteristics and advantages of the invention will become better apparent from the following detailed description of a preferred but not exclusive embodiment of a method for selective detection, with a thermographic plate, of traces of blood flow that differ from traces with correct course in vessels at different depths, particularly of the breast, and of an apparatus for performing the method and for generating thermographic maps of blood flow according to the invention, illustrated by way of non-limiting example in the accompanying drawings, wherein:

FIGS. 1a and 1b are schematic views of a method for selective detection, with a thermographic plate, of traces of blood flow that differ from traces with correct course in vessels at different depths, particularly of the breast;

FIG. 2a is a side view and FIG. 2b is a front perspective view of an apparatus for performing the method and for generating thermographic maps of blood flow;

FIG. 3 is an exploded perspective view of the apparatus according to the invention;

FIG. 4 and FIG. 5 are front perspective views of the front end of the apparatus according to the invention, respectively without and with the film supporting frame;

FIG. 6 is an exploded front perspective view of the components of the frame for insertion of the thermographic plate;

FIG. 7 is an exploded perspective view of the frame of the thermographic plate.

### DESCRIPTION OF THE PREFERRED EMBODIMENTS

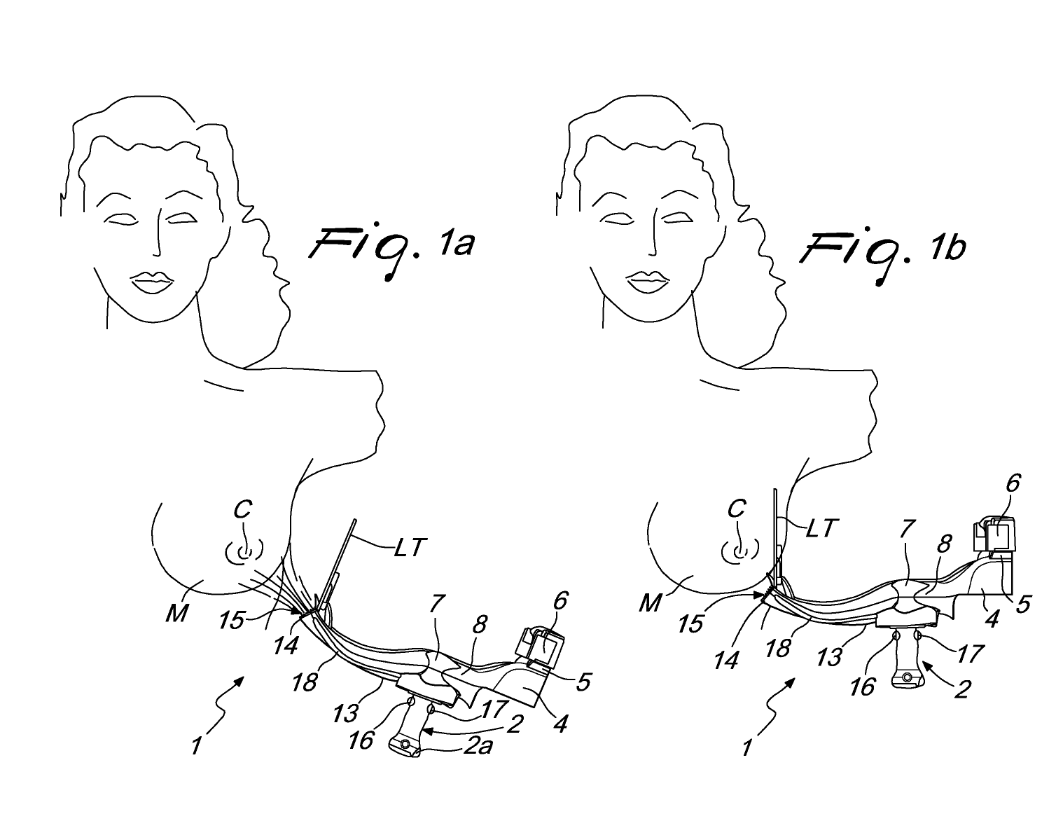

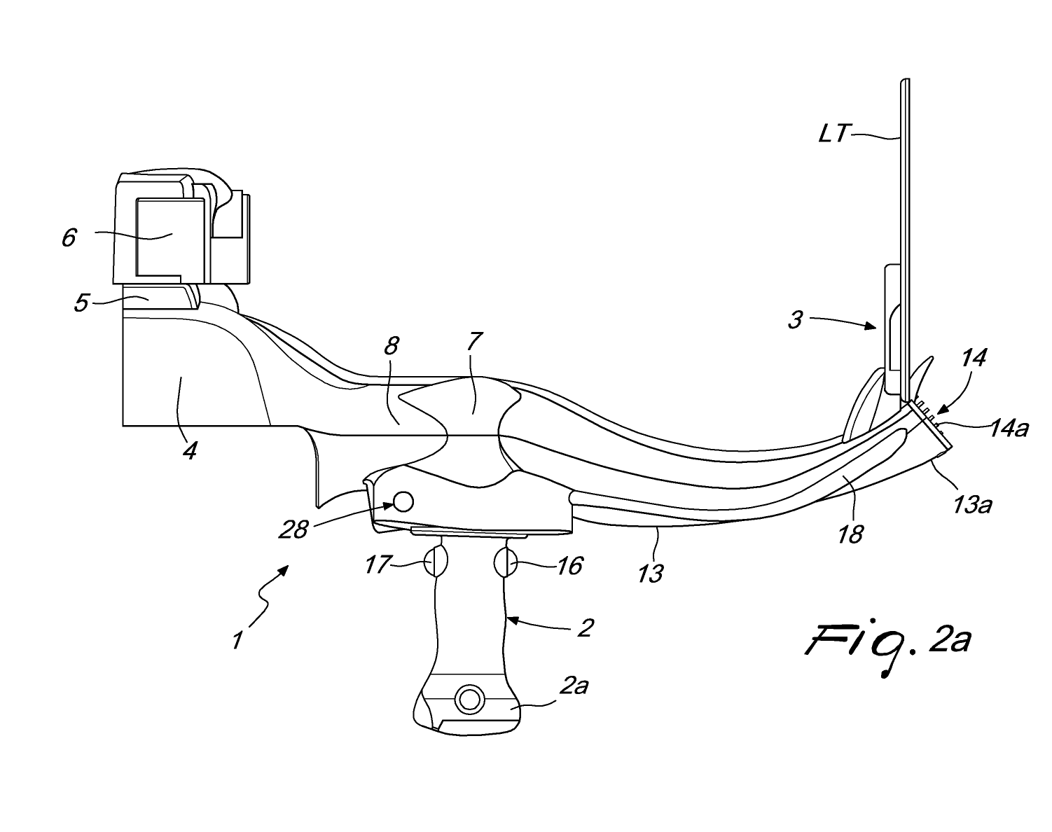

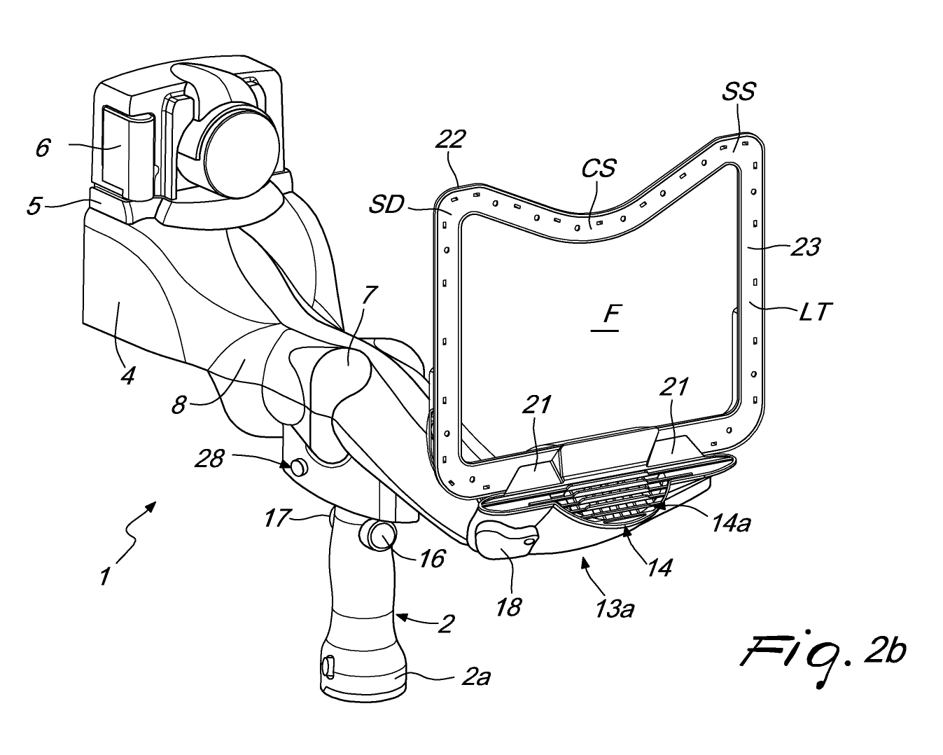

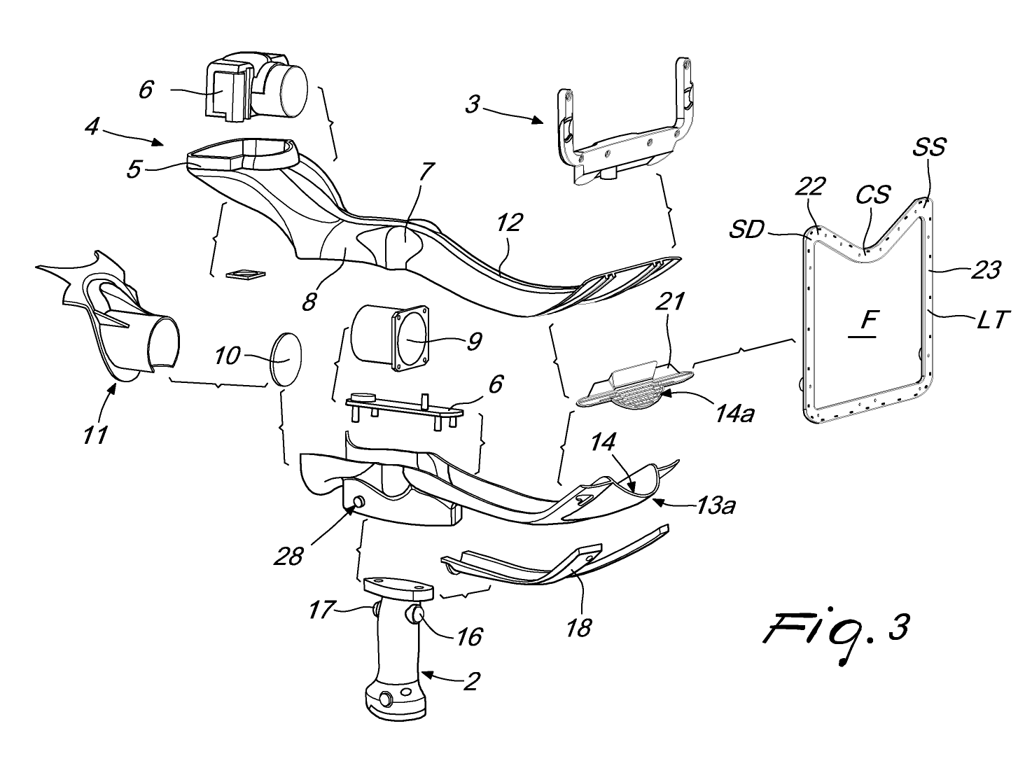

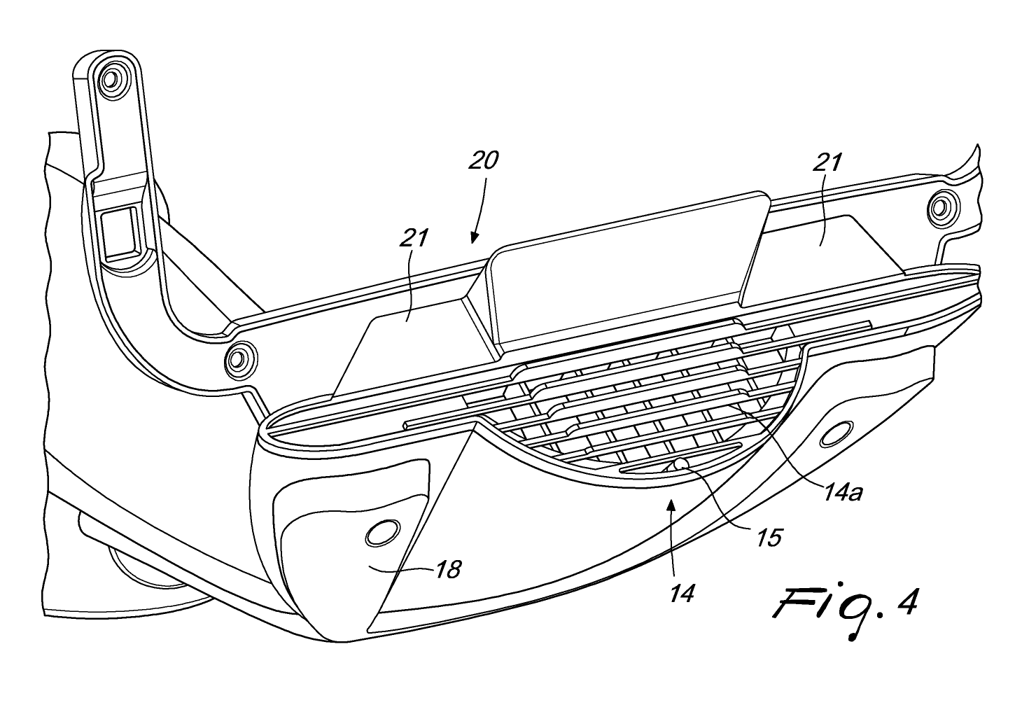

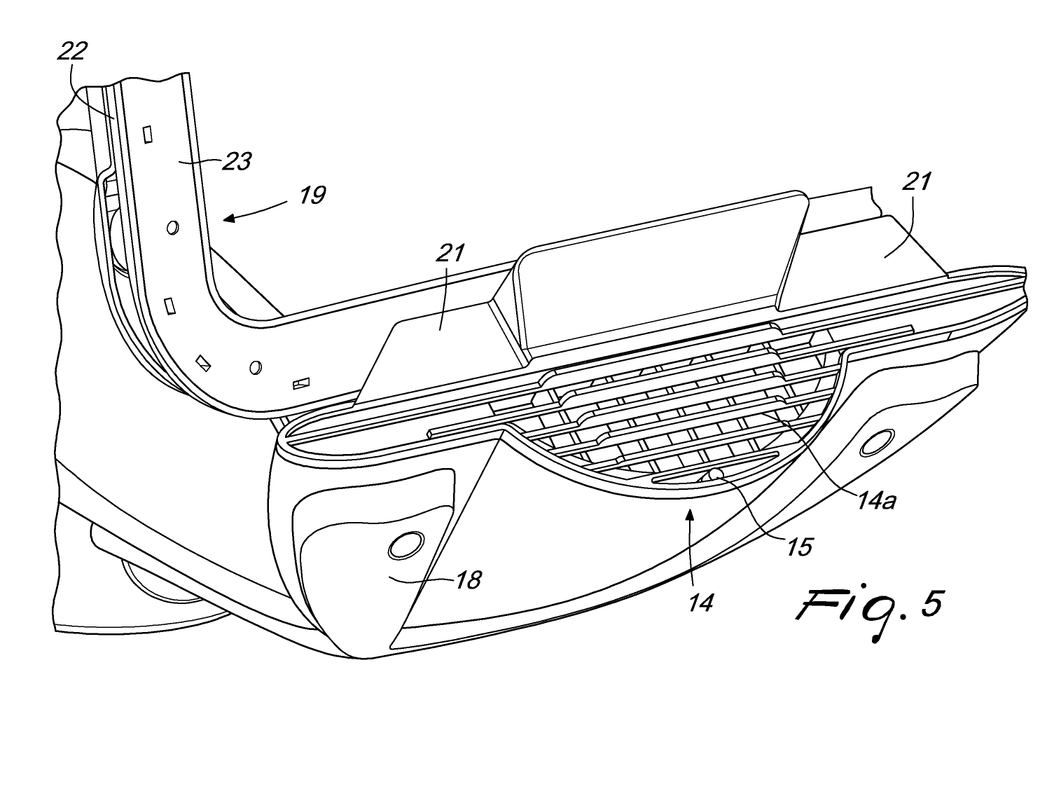

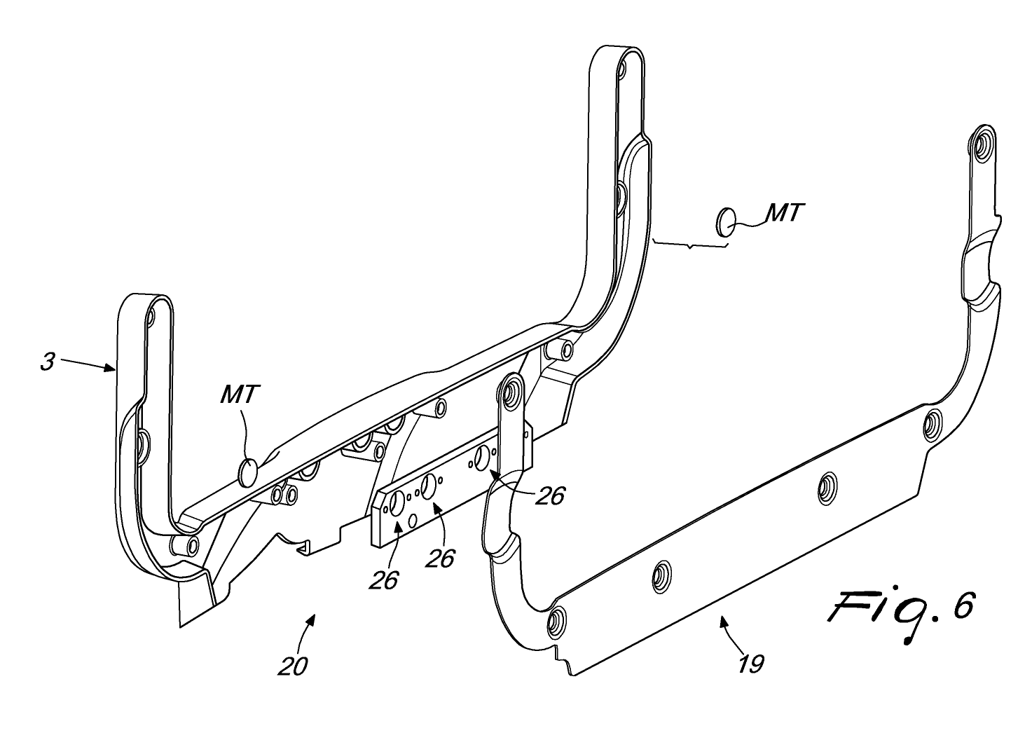

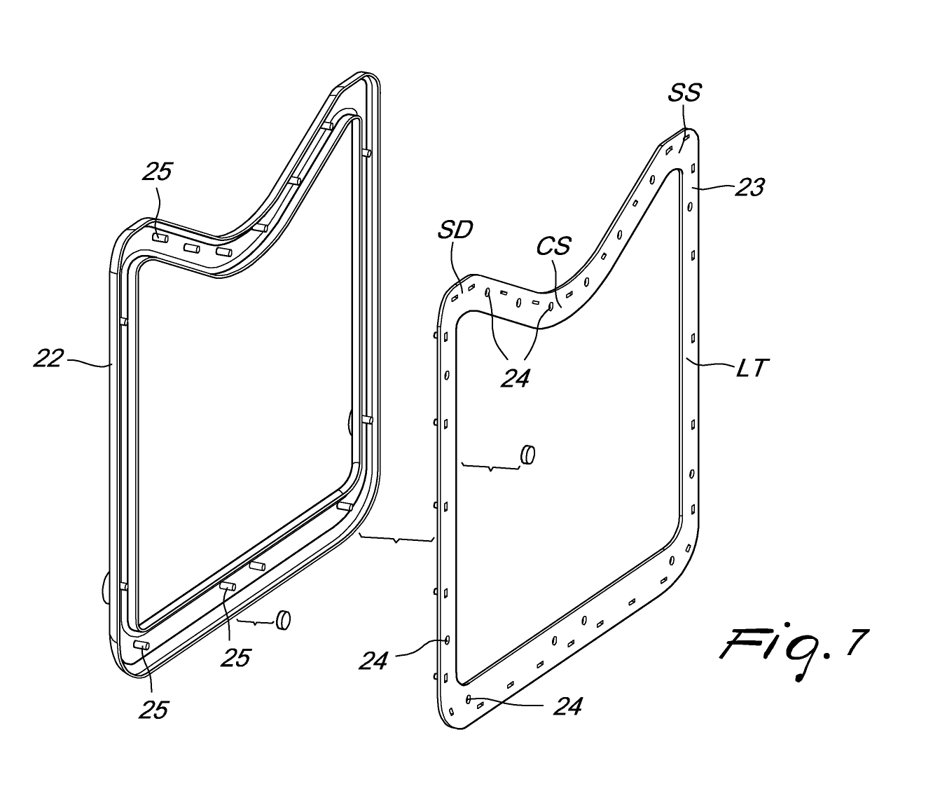

With reference to the figures, FIG. 1 illustrates schematically a method for selective detection, with a thermographic plate LT, of traces of blood flow that differ from traces with correct course in vessels at different depths, particularly of the breast M: a sequence of thermographic images is taken, such images being acquired by the thermographic plate LT which is gently pressed against the skin, which, prior to taking of each image, is cooled with quick air jets whose duration is correlated to the depth of the portion of vessel being investigated; the traces of blood vessels with correct course have orientations which are substantially centripetal with respect to the breast and taper toward the center C of the organ to confirm a blood flow temperature that decreases because the vessel gradually branches, becoming thinner, and because the depth from the surface of the skin increases.

The apparatus for performing the method of FIG. 1 comprises an elongated stand 1, which is hollow and contoured so as to convey longitudinally a stream of air, and is provided with a handle 2 and, at its two ends, with a frame 3 on the front for the snug insertion of a thermographic plate LT and on the rear with supporting means 4, which advantageously consist of a bracket 5 for resting the base of a camera MF and of a screw for coupling, by screwing in a threaded hole, the base of the camera: the base 2a of the handle is advantageously contoured and is provided with power supply laminae for coupling to the power supply of the batteries of the apparatus.

The stand 1 is provided by molding materials such as plastics and comprises an upper half-shell 7, which runs along the entire length of the stand and forms the bracket 5 on the rear, forms a box-like seat 8 in a substantially central position in which the concavity is directed downward for the placement of a support 6 for the electronic components and for the interlocking coupling of a tubular duct 9, in which an air blowing fan is accommodated with a corresponding electric motor and a filter 10, which are fitted in a lower cover for closing the rear end of the half-shell 7; in the front, the concave seat 8 of the half-shell 7 is flattened and forms a sort of wave 12, which is elongated longitudinally and has a flattened end section.

A lower half-shell 13 is fixed below the half-shell 7 and has, in the front region, a shape which is complementary to the wave 12 and forms, in cross-section, a central concavity 13a, which diverges upward and cooperates with the flat end section of the upper half-shell to form an outlet 14 for the outflow of the air jets which is central and concentrated, such outlet being located in front of the base and at the center of the frame and being protected by a grille 14a; the selective succession of the images taken after the respective blowing actions is considered independently of the colors assumed by the plate and as a function of the geometry of the traces, which are adapted to highlight the variation of the flow-rate of blood in the blood vessel.

The handle 2 is of the pistol type and is provided with a first trigger 16 for activating the motor of the fan and with a second trigger 17, which is operatively connected to the shutter release button of the camera: at the base of the air outflow duct there is a lamp 15, advantageously of the type known by the acronym LED, for lighting the area struck by the air stream: the lamp 15 also is activated by the first trigger 16.

The first and second triggers are located respectively at the front and at the rear on the top of the handle 2 to be actuated respectively with the index finger and with the thumb.

A contoured flat stiffening fork 18 is fixed below the front portion of the lower half-shell.

The frame 3 consists of two brackets 19 and 20, respectively on the front and on the rear, which are U-shaped and flattened and form, at their sides, two lateral clamps whose tips are drawn together by magnetic pellets 21 for coupling from above the two sides of the thermographic plate LT by sliding with snug fitting.

The plate LT is substantially rectangular and consists of a front frame 22 and a rear frame 23, which are provided with holes and pins 24, 25 for being fixed to each other and with the right upper corner SD and the left upper corner SS and the upper side LS which are curvilinear, respectively convex and concave, and allow access to locations of the body, for example the armpit, which would not be accessible with a straight upper side and with sharp upper corners.

The method according to the invention comprises in succession the step of activating the fan motor to blow quick jets of air against the skin region, the step of resting the thermographic plate against the skin region, the step of taking at least one photograph of the thermographic plate with the camera and the step of sorting the photographic images so that they represent, by means of groups of successive images, blood flow in the various vessels.

The thermographic plate comprises a thin support, which is stretched between the two frames 22, 23, has a thickness comprised between two and eight hundredths of a millimeter and is advantageously made of cellophane or of a material known by the trade-name Mylar.

At the base and at the center of the frame 3, in the portion of the bracket 20 that is directed toward the camera, it is possible to arrange in a horizontal row abbreviated codes 26, which can be selected among a series of indicator disks, which indicate at a glance the type of photograph, the body part involved, and other information related to the photograph; as an alternative, the codes can be selected and listed by means of a button 28, which is located above the pistol-grip handle, in a preset position of the photograph.

At the base of the bracket 19 there is a tab 27, which forms a pocket for the insertion of the base of the frame of the plate LT from above.

The plates used in the past were of a rather flexible, deformable and heat-sensitive type, whereas the ones currently used are more rigid.

The operation of the invention is relatively simple. By means of successive quick air jets, the skin of the breast is cooled and the thermographic plate is rested against the breast, pressing gently; the lines of the blood vessels revealed in a healthy breast comprise elongated traces of a certain width, which are directed substantially centripetally with respect to the breast and toward the center of the breast taper slightly and form a tip which substantially indicates that the blood vessel being investigated has branched or that its distance from the surface of the skin has increased.

If truncated or ball-shaped or crescent-like traces, or traces that branch into two or three lines appear instead of the correct elongated and pointed traces, with images that persist even despite selective cooling, this is an indicator of the presence, in the breast, of regions with increased blood inflow.

Using additional quick air jets, it is possible to cool the tissues at greater depth and follow the blood flow in the main vessels in order to check that there is no sudden reduction in blood flow, a non-pointed vessel trace, indicating that in that specific region of the breast the tissues demand a greater supply of blood in a situation that normally precedes the formation of tumors.

A time marking device is advantageously installed in the frame and records the overall duration of the cooling jets and prints it on the thermographic plate in a preset position.

Blood flow traces may also occur in regions that do not correspond to the main vessels; such traces, which are not pointed, indicate the formation of new vessels suitable to supply tumoral lesions. Advantageously, two series of thermographic recordings are made, one with the thermographic plate that reacts at a temperature of 32° C. and the other one at 33° C.; the series conveniently consist of four views each, namely Right Lateral, Right Frontal, Left Frontal, Left Lateral; the Lateral/Frontal views and the different sensitivity of the plates are adapted to provide a spatial representation of the vessels.

The thermographic investigation according to the invention has proved to be effective when the investigation is performed on a patient for the first time; the investigation is far more effective if it is possible to compare it with a similar investigation performed earlier: in this case it is possible to note that in the absence of neotumoral formations, the blood flow in the various vessels remains the same, whereas even small variations in the traces of blood flow in some vessels indicate an increased blood supply activity for the tissues to be considered as alarming.

The method and the apparatus according to the invention have yielded extremely interesting results even in a young female population, under 40 years of age, a population for which known investigation systems are unable to report situations of concern.

With the method and the apparatus according to the invention it is possible to identify a lobular tumor, which is generally an occasional finding, since it is not involved in the formation of micro-calcifications or lacks of organization that are clearly visible with a mammogram: however, the lobular tumor, by having a microcirculation that is similar to that of the ductal tumor, can be identified easily with the method and the apparatus according to the invention.

It is noted that the method and the apparatus according to the invention work non-invasively, without emitting radiation and without the injection of contrast liquids: therefore, it is possible to perform the investigation whenever one wishes, even a short time after an investigation of any type.

It has thus been shown that the invention achieves the intended aim and objects.

The invention thus conceived is susceptible of numerous modifications and variations, all of which are within the scope of the appended claims.

All the details may further be replaced with other technically equivalent ones.

In the exemplary embodiments shown, individual characteristics, given in relation to specific examples, may actually be interchanged with other different characteristics that exist in other exemplary embodiments.

Moreover, it is noted that anything found to be already known during the patenting process is understood not to be claimed and to be the subject of a disclaimer.

In practice, the materials used, as well as the shapes and dimensions, may be any according to requirements without thereby abandoning the scope of the protection of the appended claims.

The disclosures in Italian Patent Application No. BO2008A000673 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1-11. (canceled)

12. A method for selective detection, with a thermographic plate, of traces of blood flow that differ from traces with correct course in vessels at different depths, particularly of the breast, wherein a sequence of thermographic images is taken which are acquired by a thermographic plate gently pressed against a skin region, which is cooled before each photograph with quick air jets whose duration is correlated to the depth of the portion of vessel being investigated, the traces of vessels with correct course having a substantially centripetal direction with respect to the breast and tapering toward the center of the organ so as to confirm a temperature of the blood flow which decreases because the vessel gradually branches and tapers and because the depth from the surface of the skin increases.

13. The method according to claim 12, further comprising in succession the steps of: activating a fan motor to blow a jet of air against said skin region; resting said thermographic plate against said skin region; taking at least one photograph of said thermographic plate with a camera; and sorting the photographs so as to represent with groups of successive images the blood flow in the various vessels.

14. The method according to claim 13, wherein two series of thermographic recordings are made, one with a thermographic plate that reacts at a temperature of 32° C. and one at 33° C., presenting four views each, namely Right Lateral, Right Frontal, Left Frontal, Left Lateral, the Lateral/Frontal views and the different sensitivities of the plates being adopted to provide a spatial representation of the vessels.

15. An apparatus for performing a method for selective detection, with a thermographic plate, of traces of blood flow that differ from traces with a correct course, comprising a stand provided with a handle which has, at its two ends, a frame for the snug fit of a thermographic plate and supporting means for a camera, further comprising a cooling fan associated with said stand, which is mounted and actuated so that it can rotate in a duct whose outlet has a reduced area and is located in the front of the base and at the center of said frame, the selective succession of the images taken after the respective jets being considered independently of the colors assumed by the plate and as a function of the geometry of the traces, which are suitable to highlight the variation of the blood flow-rate in said vessel.

16. The apparatus according to claim 15, wherein said handle is of the pistol type and in that there is a first trigger for activation of the motor that drives the fan and a second trigger that is functionally connected to a shutter release button of said camera.

17. The apparatus according to claim 16, wherein said first and second triggers are located respectively on the front and on the rear at the top of said handle to be operated respectively with the index finger and with the thumb.

18. The apparatus according to claim 15, wherein said thermographic plate has a substantially rectangular shape, in which the upper corners and the upper side are curvilinear and respectively convex and concave and are adapted to allow access to particular locations of the body that are problematic to access, for example the armpit.

19. The apparatus according to claim 15, wherein said thermographic plate comprises a thin support, whose thickness is comprised between two and eight hundredths of a millimeter.

20. The apparatus according to claim 19, wherein said thin support of said thermographic plate is made of cellophane or of a material known by the trade-name Mylar.

21. The apparatus according to claim 15, wherein at the base of said frame there are, in a horizontal row, abbreviated codes which can be selected among a series of indicator disks, which are adapted to indicate at a glance information related to the photograph.

22. The apparatus according to claim 15, wherein a time marking device is installed in the frame and records the overall duration of the cooling jets and prints it on the thermographic plate.
